(19) United States
(12) Patent Application Publication
(10) Pub. No.: US 2025/0221732 A1
(43) Pub. Date: Jul. 10, 2025

(54) MEDICAL PUNCH TIP, SURGICAL INSTRUMENT AND METHOD

(71) Applicant: KAMMERER MEDICAL SYSTEMS GMBH & CO. KG, Stockach (DE)

(72) Inventors: Rüdiger SCHWÄGLER, Stockach (DE); Helena SCHREMPP, Stockach (DE)

(21) Appl. No.: 18/853,017

(22) PCT Filed: Mar. 30, 2023
(86) PCT No.: PCT/DE2023/100248

§ 371 Date: Sep. 30, 2024

(30) Foreign Application Priority Data
Mar. 31, 2022 (DE) 10 2022 107 752.7

(51) Int. Cl. A61B 17/3205; A61B 17/00

(52) U.S. Cl. CPC A61B 17/32053; A61B 2017/00473; A61B 2017/00477

(57) ABSTRACT

A medical punch tip may include a first tip part and a second tip part which is longitudinally movably mounted on the first tip part. The second tip part is non-removably connected to the first tip part without tools.

### FIELD

The present disclosure concerns a medical punch tip for an actuation device, a surgical instrument and a method for detaching a medical punch tip from an actuation device.

### INTRODUCTION

In some medical interventions, surgical instruments are used to remove or extract bones, cartilage or other tissue. Such instruments usually have a longitudinal shank on which a medical tool is provided at the distal end, i.e. the end remote from the user. The user can actuate the tool, e.g. a punch, via a mechanism at the proximal end of the shank, i.e. the end nearest the user.

Because such punches rapidly become blunt during a medical intervention or are required in different sizes, surgical instruments with interchangeable punch tips have been developed. Depending on requirements therefore, the desired or “fresh” punch tips can be attached successively to a single actuation device, which is held by the user and the proximal end of which comprises the mechanism for actuating the tool.

These punch tips usually have a first tip part and a separate second tip part. At a distal end, i.e. the end remote from the user or the actuation device, the first tip part has a cutting plate also known as an “anvil”, against which a U-shaped cutter at the distal end of the second tip part cuts on a longitudinal displacement of the two tip parts relative to one another. The first tip part is usually attached to a base body of the actuation device during a first attachment step. The second tip part must be attached separately, in a second attachment step, to a slide which is movable relative to the base body.

In view of the stress and pressure of time normally associated with such medical interventions, such an attachment of the tip parts has however proved to be too complex. There is also the risk that attempts will be made to mount the two parts incorrectly, e.g. the wrong way round, or the tip parts are incorrectly swapped.

### SUMMARY

In this context, it is an object of the present disclosure to improve the user-friendliness during handling of a surgical punch instrument or at least parts thereof.

This object is achieved by a medical punch tip, a surgical instrument and a method for detaching a medical punch tip from an actuation device according to the independent claims.

Preferred embodiments are the subject of the dependent claims and the description which follows.

A first aspect of the present disclosure relates to a medical punch tip for an actuation device. The punch tip has a first tip part and a second tip part which is mounted longitudinally displaceably on the first tip part. According to the present disclosure, the second tip part is toollessly inseparably connected to the first tool part.

An aspect of the present disclosure is based on the approach of providing a toollessly inseparable connection of two tip parts of a medical punch tip which can be attached, i.e. connected, to a corresponding actuation device. In contrast to conventional punch tips, in which tip parts can be attached to and/or detached from such an actuation device independently of one another, the punch tip according to the present disclosure may allow a particularly rapid and simple change of tool tip, in particular because the two tip parts can only be attached to and/or detached from the actuation device together because of the toollessly inseparable connection. For example, the punch tip may be attached to and/or detached from the actuation device with particularly few manual actions. Because of the toollessly inseparable connection, the attachment and/or detachment of the first tip part to or from the actuation device, such as a base body of the actuation device, also causes a corresponding attachment or detachment of the second tip part to or from the actuation device, in particular a slide of the actuation device. Conversely, naturally also an attachment and/or detachment of the second tip part to or from the actuation device may cause a corresponding attachment or detachment of the first tip part to or from the actuation device. This may be advantageous in particular during a medical intervention, with respect to the then prevailing sterile conditions and the stress associated with such an intervention.

Because of the toollessly inseparable, i.e. permanent connection of the two tip parts, an improved handling during cleaning and/or sterilization of the punch tip is also possible. In particular, the loss of one of the two tip parts may thus be excluded. At the same time, the permanent connection of the tip parts may reduce the stock level, and in some cases also the space required.

The toollessly inseparable connection may also help ensure that the punch tip cannot be attached to an actuation device incorrectly, e.g. the wrong way round. In particular, such permanently connected tip parts may counter a confusion of tip parts.

In the development of the punch tip according to the present disclosure, it was found in particular that also two tip parts, which are toollessly inseparably connected together but mounted longitudinally displaceably on one another, can be cleaned and/or sterilized effectively and thoroughly. Surprisingly, tests have shown that cleaning fluid can for example also penetrate into a form-fit connection of the tip parts, i.e. into corresponding gaps between the two tip parts. In addition, because the two tip parts are longitudinally displaceable relative to one another, different portions of the punch tip are always exposed so that, overall, a level of cleanliness and/or sterility of the tool tips can be achieved which meets the requirements of medical use.

Preferred embodiments of the present disclosure and their refinements are described below, which may be combined arbitrarily with one another and with other aspects of the present disclosure described below, unless expressly specified otherwise.

In some preferred embodiments, the first tip part has a first attachment portion for in particular toollessly separable attachment, preferably by form fit, of the first tip part to a base body of the actuation device. Alternatively or additionally, the second tip part has a second attachment portion for in particular toollessly separable attachment, preferably by form fit, of the second tip part to a slide of the actuation device. The slide is suitably longitudinally displaceably attached to the base body so that the slide can be longitudinally displaced relative to the base body by actuation of the actuation device. Thus the second punch tip is also longitudinally displaceable relative to the first punch tip.

For example, the first tip part may have a first attachment portion which in particular may be received by form fit by the base body, for example in a base body attachment portion, e.g. in a corresponding base body recess. Alternatively or additionally, the second tip part may have a second attachment portion which in particular may be received by form fit by the slide, for example in a slide attachment portion, e.g. in a corresponding slide recess. By means of such attachment portions, separate and/or additional attachment mechanisms, which in some cases require additional components and could be undesirably actuated, may be omitted.

In a preferred embodiment, the first attachment portion and the second attachment portion have at least partially substantially the same contour. In other words, the first tip part in the first attachment portion and the second tip part in the second attachment portion are preferably configured such that, at least partially and from a predefined viewing angle, they have the same outline. The first and second attachment portions need not necessarily have the same dimensions. In particular, it is conceivable that the first attachment portion is formed at least partially smaller, in particular narrower, than the second attachment portion. The matching contours of the two attachment portions allows simple and intuitive handling of the punch tip, in particular on detachment from an actuation device. The attachment portions may in particular be brought into “congruence” by the matching contours, and thus be removable from a corresponding recess in the actuation device.

To this extent, it is preferred that, for toolless detachment of the punch tip from the actuation device, the second tip part can be brought into a detachment position by longitudinal displacement relative to the first tip part. Preferably, the contour of the second attachment portion then at least partially substantially aligns with the contour of the first attachment portion. This for example allows the first attachment portion together with the second attachment portion to be removed from a recess formed in the actuation device. In particular, the first attachment portion may thus be conducted through a slide attachment portion of the slide, which is actually configured to receive the second attachment portion by form fit.

In the sense of the present disclosure, two components or their contours here in particular at least partially substantially align with one another when the essential features of the components or contours, such as corners or edges or other significant protrusions, are oriented to one another. The term “align” thus does not mean absolute congruence. Rather, it may mean a relative position of two components in which the components or their contours, in particular their significant portions, correspond to one another. To this extent, two components or their contours may at least partially substantially align with one another if the one component is dimensioned slightly larger than the other component.

An at least partial substantial alignment of the contours of the first attachment portion and second attachment portion is possible for example if the first tip part has a top side facing the second tip part with a flat first sliding face. Accordingly, the second tip part may have an underside facing the first tip part with a flat second sliding face, via which the second tip part slides along the first tip part during a longitudinal displacement, for example until the detachment position is reached. This may facilitate smooth operation of the punch tip.

In order for example not to hinder a detachment of the first tip part from the base body by the slide attachment portion, it is preferred that the first sliding face and the second sliding face are at least partially similarly formed. The term “similarly formed” may here in particular mean a form of two sliding faces in which the sliding faces have the same shape. The same dimensions, i.e. the same sizes, are not absolutely necessary. In particular, the first sliding face may be smaller than the second sliding face. The matching contours of the two attachment portions can be created by means of sliding faces formed in this way.

In a further preferred embodiment, the first tip part has a first protrusion which, running longitudinally, adjoins a proximal end of the first tip part. Alternatively or additionally, the second tip part has a second protrusion which, running longitudinally, adjoins a proximal end of the second tip part. A particularly robust connection of the tool tip to the actuation device is possible by means of such protrusions. In particular, this may prevent the punch tip from falling out of the actuation device during use when the latter is rotated by 180° about its longitudinal axis. Also, the tool tip may thus be connected to the actuation device without formation of a step.

The first protrusion suitably has a top side with a flat top side face. Alternatively or additionally, the second protrusion has an underside with a flat underside face. Preferably, the underside face is oriented substantially parallel to the top side face. With such a design of the first and second protrusions, a smooth actuation of the tool tip can be ensured.

A particularly secure and form-fit attachment of the tool tip to the actuation device can be achieved if the first and second protrusions preferably have respective side walls, which run substantially perpendicularly to the top side face and underside face and are formed similarly uneven.

For example, the first tip part in the first attachment portion may have a top side with a top side face and a first bulge, which runs transversely to a surface normal of the top side face. Alternatively or additionally, the second tip part in the second attachment portion may have an underside with an underside face facing the top side face and a second bulge, which runs transversely to a surface normal of the underside face and is formed similarly to the first bulge. Here too, the term “formed similarly” means in particular that the forms have the same shape, e.g. the same contour. The bulges need not however necessarily be made the same size. The bulges allow a form-fit attachment to the actuation device, in particular an undercut by the base body attachment portion or slide attachment portion. By means of the similar bulges, the punch tips can also be reliably removed from the actuation device. If these are arranged substantially aligned with one another, it is possible to conduct the first attachment portion out through a recess in the slide, in which the second attachment portion is arranged during operation.

Suitably, the first tip part and/or the second tip part each have a convex curvature in the region of the respective first or second bulge. Thus in particular, in the slide attachment portion, the slide may be bent when pushed onto the second tip part, so that the second attachment portion can slide into the slide and a part of the slide attachment portion can engage behind the second bulge.

In a further preferred embodiment, the first tip part has a first step in the first attachment portion. The step serves preferably as a stop for the second tip part during a longitudinal displacement. In other words, the step may limit the displacement travel of the second tip part. Also by means of the step, in particular it may be prevented that the second tip part can be withdrawn completely from the first tip part by longitudinal displacement. To this extent, the first step preferably constitutes a reliable means, which is simple to implement with respect to production, for toollessly inseparable connection of the first tip part to the second.

It is suitable if the first step is arranged at a proximal end of the first attachment portion, i.e. an end nearest to a user on connection to the actuation device or during use. Thus the displacement travel may be maximized.

In order to allow an alignment of the contours of the first and second attachment portions in the detachment position, it is preferred that the second tip part in the second attachment portion has a second step formed correspondingly to the first step. The second step is here suitably provided, e.g. formed, such that a proximal end of the first attachment portion substantially aligns with a proximal end of the second attachment portion when the second tip part stops on the first step.

The first and second steps are suitably configured not only to transfer the second tip part to the detachment position. Rather, it is preferred that the first and second steps, additionally or at least alternatively, form a centering device. Suitably, in particular quite generally, a centering device is provided which is configured to orient the second tip part relative to the first tip part when the second tip part is brought into the detachment position by longitudinal displacement relative to the first tip part, for detachment from the actuation device. This may significantly facilitate the detachment of the punch tip from the actuation device. In particular, in this way a lateral play of the second tip part relative to the first tip part can be at least reduced.

A reliable and precise centering of the second tip part relative to the first tip part may be achieved for example if the first step is formed as a disk. Suitably, the second step is formed with a correspondingly curved edge. When the first and second steps are brought into contact, the second tip part is thus automatically oriented to the first step.

In a further preferred embodiment, the second attachment portion protrudes laterally on both sides over the first attachment portion transversely to a longitudinal extent of the punch tip. In other words, the first tip part in the first attachment portion is formed narrower than the second tip part in the second attachment portion. This may ensure that only the first tip part can be attached to the base body, and not the second tip part. In particular, it may thus be prevented that the punch tips are attached to the actuation device incorrectly, in particular the wrong way round, i.e. the second tip part attached to the base body.

As an alternative to the different dimensioning, in particular width, of the first and second attachment portions, other means for preventing incorrect attachment are also conceivable. For example, on a side opposite the second tip part, the first tip part may have a groove for receiving a guide rail of the base body. Thus the first and second attachment portions can only be completely received by the actuation device when the groove faces the guide rail. To this extent, in some embodiments, the tool tips may quite generally be configured to prevent incorrect attachment to the actuation device.

In a further preferred embodiment, the punch tip is configured as a sterile single-use tool. This may substantially simplify the logistics, e.g. in a hospital, since the tool tip need not be sterilized after use. Also, in this way new and hence sharp tool tips can always be provided. Because of the guaranteed sterility of such a single-use tool, patient safety may also be increased.

Alternatively however, it is also possible that the tool tip is formed to be reusable, i.e. in particular cleanable and/or sterilizable. This may in some cases reduce the complexity of procurement and/or costs.

A second aspect of the present disclosure concerns a surgical instrument with a medical punch tip according to the first aspect of the present disclosure, and an actuation device for actuation of the punch tip. Such an instrument can be operated easily and safely, in particular because of the permanent connection of the first and second tip parts. The reusability of at least the actuation device may also save raw materials, storage space and energy. To this extent, the instrument is particularly environmentally friendly and economic.

A third aspect of the present disclosure concerns a method for detaching a medical punch tip, in particular according to the first aspect of the present disclosure, from an actuation device with a base body on which a first tip part of the punch tip is attached, and a slide which is attached longitudinally displaceably on the base body and on which a second tip part of the punch tip is attached, which is mounted longitudinally displaceably on the first tip part and hence toollessly inseparably connected thereto. In the method, the second tip part is brought into a detachment position relative to the first tip part by longitudinal displacement of the slide relative to the base body, and the punch tip is then laterally withdrawn from the actuation device. In other words, after creation of the detachment position, the first and second tip parts are together laterally withdrawn from the actuation device. Suitably, the first attachment portion of the first tip part is conducted out of the base body attachment portion of the base body through the slide attachment portion of the slide.

The term “laterally withdrawn” in the sense of the present disclosure may mean withdrawal transversely to a longitudinal extent of the punch tip and/or the actuation device, i.e. in particular transversely to the movement direction of the second tip part during longitudinal displacement relative to the first tip part. For example, on a lateral withdrawal, the attachment portions of the first and second tip parts may be extracted from the actuation device, in particular from the base body and slide.

This method allows a rapid exchange of punch tips even under stress and in sterile conditions, for example in the operating theater. This allows shorter operation times. Here, it may be avoided that individual parts of the punch tip can become detached from one another and are lost.

The present disclosure is explained in more detail below with reference to the figures. Where appropriate, elements with the same function carry the same reference signs. The present disclosure is not restricted to the exemplary embodiments illustrated in the figures—also not in relation to functional features. The above description and the following description of the figures contain numerous features which may be partially combined in the dependent subclaims. These features, and all other features disclosed above and in the following description of the figures, may also be considered individually by the person skilled in the art and combined into suitable further combinations. In particular, all cited features may be achieved individually and in arbitrary suitable combination with the medical punch tip according to the first aspect of the present disclosure, the surgical instrument according to the second aspect of the present disclosure, and the method according to the third aspect of the present disclosure.

### BRIEF DESCRIPTION OF THE DRAWINGS

The drawings show, at least partially schematically:

FIG. 1 an example of a surgical instrument;

FIG. 2 components of the surgical instrument from FIG. 1;

FIG. 3 an example of a medical punch tip in a side view;

FIG. 4 the medical punch tip from FIG. 3 in exploded view;

FIG. 5 the medical punch tip from FIG. 3 in a top view;

FIG. 6 an example of a distal end of an actuation device in a side view; and

FIG. 7 an example of a method for detaching a medical punch tip from an actuation device.

### DETAILED DESCRIPTION

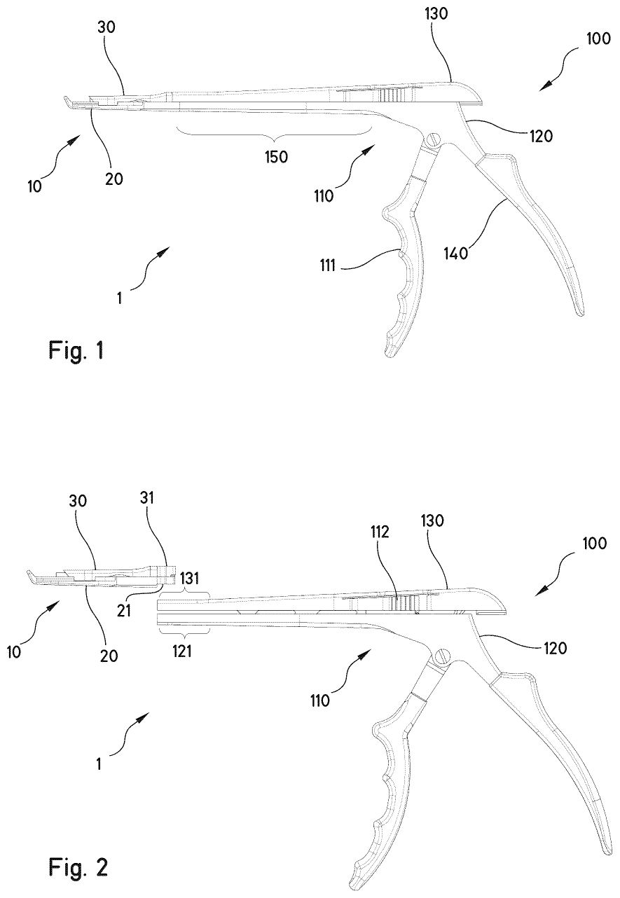

FIG. 1 shows an example of a surgical instrument 1 with a medical punch tip 10 and an actuation device 100. The punch tip 10 has a first tip part 20 and a second tip part 30 which is mounted longitudinally displaceably on the first tip part 20. The actuation device 100 has a base body 120 and a slide 130 which is attached to the base body 120 so as to be at least partially longitudinally displaceable. The longitudinal displacement of the slide 130 relative to the base body 120 may be triggered for example by means of an actuation mechanism 110.

At a proximal end, i.e. the end nearest a user during use of the instrument 1, the actuation device 100 preferably has a handle 140. It is suitable if the handle 140 is formed by the base body 120. Another part of the base body 120, together with a corresponding portion of the slide 130, forms a shank 150.

The punch tip 10 is arranged at a distal end, i.e. an end remote from the user during use of the surgical instrument 1, of the actuation device 100, in particular the shank 150. The punch tip 10 is exchangeable (see FIG. 2) and in the present illustration is connected to the actuation device 100 ready for operation. Suitably, for this the first tip part 20 is attached to the base body 120 and the second tip part 30 is attached to the slide 130. For example, because of a shorter design of the second tip part 30 relative to the first tip part 20, in this actuation position the slide 130 protrudes over the base body 120 at the distal end of the actuation device 100.

When the actuation mechanism 110 is triggered, in particular by pulling of a trigger lever 111, the slide 130 moves relative to the base body 120 towards the distal end. Because of the connection of the slide 130 to the second tip part 30, the punch tip 10 may thus be actuated.

FIG. 2 shows components of the surgical instrument 1 from FIG. 1, namely in particular the actuation device 100 and the punch tip 10 detached therefrom. A first attachment portion 21 of the first tip part 20 and a second attachment portion 31 of the second tip part 30 are therefore visible. The second tip part 30 is in a detachment position relative to the first tip part 20, in which the punch tip 10 can be detached from the actuation device 100. Suitably, in this detachment position, the proximal ends of the first and second tip parts 20, 30 and/or the contours of the first and second attachment portions 21, 31, at least partially substantially align with one another.

Also, the slide 130 is in a detachment position relative to the first base body 120, in which the punch tip 10 can be detached from the actuation device 100. Here, suitably the distal ends of the base body 120 and slide 130 at least substantially align with one another.

The attachment portions 21, 31 suitably serve for attachment of the first and second tip parts 20, 30 to the base body 120 or slide 130. For attachment to the base body 120, the first attachment portion 21 can here preferably be inserted in a base body attachment portion 121 at the distal end of the base body. In other words, the base body 120 in the base body attachment portion 121 is suitably configured to receive the first attachment portion 21, in particular by form fit.

It is suitable if the base body attachment portion 121 is only accessible for the first attachment portion 21 when the slide 130 is in the detachment position. To this extent, it is preferred if the slide 130 secures the first attachment portion 21 in the base body attachment portion 121 in at least one operating position. Such an operating position suitably exists if the slide 130—in the state of the surgical instrument 1 ready for operation as shown in FIG. 1—protrudes at the distal end beyond the base body 120, in particular the base body attachment portion 121.

For attachment of the second tip part 30 to the slide 130, the second attachment portion 31 can preferably be inserted in a slide attachment portion 131 at the distal end of the slide 130. In other words, the slide 130 in the slide attachment portion 131 is suitably configured to receive the second attachment portion 31 in particular by form fit. The slide attachment portion 131 may in particular be configured for pushing onto, in particular engaging on, the second attachment portion 31. A connection between the slide 130 and the second tip part 30 can here preferably easily be created by the longitudinal displacement of the slide 130 relative to the base body 120, i.e. by actuation of the actuation mechanism 110.

Suitably, the slide 130 is raised from the base body 120 in the detachment position, as shown in FIG. 2. This can facilitate not only the detachment and attachment of the first tip part 20 from or to the base body 120, but also cleaning or sterilization of the actuation device 100. Therefore the attachment position may also be described as a cleaning or sterilization position. The actuation device 100 can be brought out of the operationally ready state shown in FIG. 1 into the detachment position, for example in that a locking of the actuating mechanism 110 is released and the slide 130 is manually drawn towards the proximal end relative to the base body 120. To release the locking, suitably a corresponding actuation element 112 is provided on the slide 130.

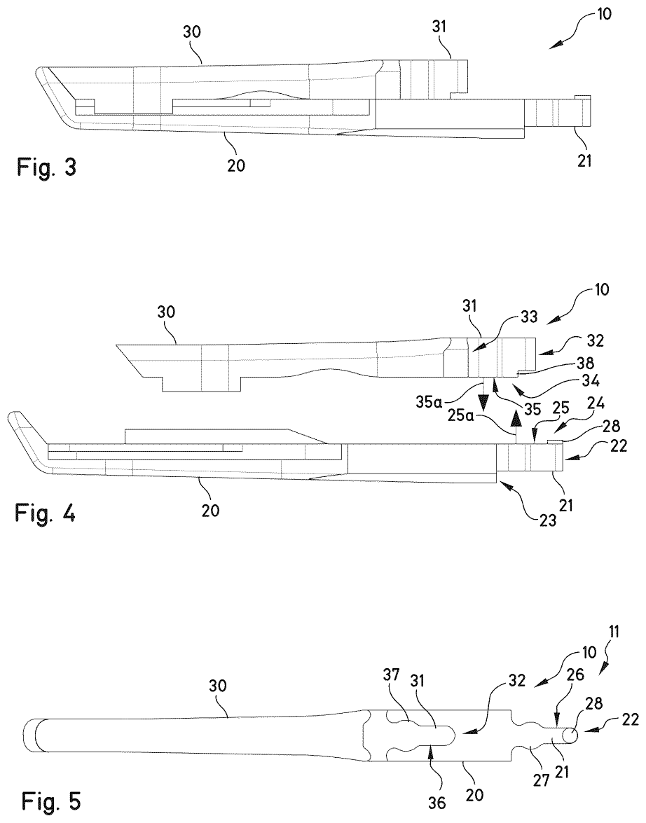

FIG. 3 shows an example of a medical punch tip 10 in a side view. The punch tip 10 has a first tip part 20 and a second tip part 30 which is mounted longitudinally displaceably on the first tip part 20. The first and second tip parts 20, 30 are here suitably toollessly inseparably connected together.

The first tip part 20 has a first attachment portion 21, and the second tip part 30 has a second attachment portion 31, which are configured respectively for attachment to a base body or slide of an actuation device (see FIGS. 1 and 2).

Suitably, the first attachment portion 21 and the second attachment portion 31 are configured, in particular arranged or able to be arranged by longitudinal displacement of the first and second tip parts 20, 30 relative to one another, such that firstly, the first attachment portion 21 can be attached to the base body and then the second attachment portion 31 can be attached to the slide. In other words, the first and second attachment portions 21, 31 may be configured, in particular arranged or able to be arranged, such that a two-stage attachment of the punch tip 10 to the actuation device is possible. For example, firstly the first attachment portion 21 may be inserted in a base body attachment portion of the base body, whereupon a slide attachment portion of the slide can be pushed onto the second attachment portion 31 by longitudinal displacement relative to the base body. In particular, by attachment of the first attachment portion 21 to the base body, the second attachment portion 31 is positioned and/or held so as to allow or at least simplify the creation of a connection between the second attachment portion 31 and the slide.

For the two-stage attachment to the actuation device, the attachment portions 21, 31 may for example be spaced from one another or be able to be spaced from one another by longitudinal displacement, in particular in a longitudinal direction of the punch tip 10. Such an arrangement may easily be achieved if the second tip part 30 is formed shorter than the first tip part 20 and can therefore be moved away from the proximal end of the first tip part 20.

Alternatively or additionally, the first attachment portion 21 and the second attachment portion 31 are configured, in particular arranged or able to be arranged by longitudinal displacement of the first and second tip parts 20, 30 relative to one another, such that the first and second tip parts 20, 30 can be detached from the base body and the slide simultaneously. In other words, the first and second attachment portions 21, 31 may be configured, in particular arranged or able to be arranged, such that a single-stage detachment of the punch tip 10 from the actuation device is possible. Such an arrangement of the first and second attachment portions 21, 31 is shown in FIG. 2. Suitably, the second tip part 30 can be longitudinally displaced relative to the first tip part 20 so far towards the proximal end that the proximal end of the second attachment portion 31 substantially aligns with the proximal end of the first attachment portion 21.

FIG. 4 shows the punch tip 10 from FIG. 3 in exploded form. Here, a possible design of the first and second attachment portions 21, 31 is shown. The first attachment portion 21 and the second attachment portion 31 may here have a respective first or second protrusion 22, 32 which, running longitudinally, adjoins a proximal end 23, 33 of the respective tip part 20, 30. In particular, the first and second attachment portions 21, 31 may be defined by the respective protrusion 22, 32. The protrusions 22, 32 are suitably formed such that, in particular, they can be received by form fit, firmly seated, by the base body in the base body attachment portion or by the slide in the slide attachment portion.

In order to allow a smooth actuation of the punch tip 10, it is suitable if the first tip part 20, in particular the first attachment portion 21, has a top side 24 with a flat top side face 25. The top side 24 suitably faces an underside 34 of the second tip part 30, in particular of the second attachment portion 31. The underside 34 suitably has a flat underside face 35.

It is preferred that the top side face 25 and underside face 35 suitably run parallel with one another. FIG. 4 shows the corresponding surface normals 25a, 35a of the top side face 25 and underside face 35 respectively.

Optionally, the first tip part 20 has a first step 28 in the first attachment portion 21. The first step 28 is preferably arranged at the proximal end of the first attachment portion 21. Suitably, the first step 28 is arranged on a side facing the second tip part 30, in particular on the top side 24. For example, the step 28 may protrude from the top side face 25. In other words, the step 28 may be formed as a protrusion on the top side 24. By means of the first step 28, a displacement travel of the second tip part 30 can be limited. To this extent, the step 28 may serve as a stop for the second tip part 30. By means of the step 28, in particular it can be prevented that the second tip part 30 is extracted from a guide of the first tip part 20 towards its proximal end 33. To this extent, the step 28 may contribute to the toollessly inseparable connection of the two tip parts 20, 30.

Alternatively or additionally, the second tip part 30 may have a second step 38 in the second attachment portion 31. The second step 38 is preferably arranged at the proximal end of the second attachment portion 31. Suitably, the second step 38 is arranged on a side facing the first tip part 20, in particular on the underside 34.

Preferably, the second step 38 is formed correspondingly to the first step 28. For example, the second step 38 may be formed in the underside face 35. In other words, the second step 38 may be formed as a recess on the underside 34. By means of the second step 38, in particular in cooperation with the first step 28, the displacement travel of the second tip part 30 can be limited. In particular, the second step 38 of the second tip part 30 can stop on the first step 28.

The first and/or second steps 28, 38 are preferably formed, in particular arranged, such that when the second tip part 30 meets the first step 28, the proximal ends 22, 32 of the first and second tip parts 21, 31 and/or attachment portions 21, 31 substantially align with one another. To this extent, by means of the first and/or second step 28, 38, the first and second tip parts 20, 30 can be oriented to one another in a detachment position, so that for example the contours of the first and second attachment portions 21, 31 at least partially substantially align with one another. This is explained in more detail in connection with FIG. 5.

FIG. 5 shows the punch tip 10 from FIG. 3 in a top view. This illustrates a possible form of the first tip part 20 in the first attachment portion 21, in particular the first protrusion 22, and the second tip part 30 in the second attachment portion 31, in particular the second protrusion 32.

The first and second protrusions 22, 32 suitably have side walls 26, 36. These side walls 26, 36 may run perpendicularly to the top side face and underside face shown in FIG. 4, i.e. perpendicularly to the drawing plane in FIG. 5. Preferably, the side walls 26, 36 are formed unevenly for reliable, in particular form-fit attachment to the actuation device.

For example, the first and second attachment portions 21, 31 may have a respective first and second bulge 27, 37. These bulges 27, 37 suitably run transversely to the surface normals of the top side face and underside face, shown in FIG. 4. In other words, the protrusions 22, 32 suitably have a lateral thickening, i.e. a thickening arranged in the region of the side walls 26, 36.

These bulges 27, 37 preferably each have a convex curvature. The first and second attachment portions 21, 31 may thus be formed such that the base body or slide can undercut the respective first or second attachment portion 21, 31. An undesirable detachment of the punch tip 10 from the distal end of the actuation device in the longitudinal direction can thereby be prevented.

As FIG. 5 clearly shows, in a possible embodiment, the first attachment portion 21 and the second attachment portion 31 have at least partially and at least substantially the same contour, in particular the same outer contour. In particular, the top side face 25 marked in FIG. 4, and the underside face 35 also marked there, may be at least partially similarly formed. To this extent, it is suitable if the side walls 26, 36 are formed similarly uneven, i.e. for example the second bulge 37 is formed at least substantially the same as the first bulge 27.

Here, it is not necessary for the contours of the attachment portions 21, 31 to be identical, i.e. to match one another without scaling. Rather, in particular the first attachment portion 21 may be dimensioned smaller, in particular formed narrower, than the second attachment portion 31. It is however preferred that the contours are similar, i.e. at least partially match one another with corresponding slight scaling.

In the case of a two-stage attachment of the punch tip 10 to the actuation device, such a matching of the first attachment portion 21 and the second attachment portion 31 allows the first attachment portion 21 to be secured in the base body attachment portion, and the slide attachment portion to be pushed onto the second attachment portion 31, by a single actuation of the actuation device. A smaller dimensioning of the first attachment portion 21 can ensure that this can easily be conducted through the slide attachment portion.

Alternatively or additionally, such a matching of the first attachment portion 21 and the second attachment portion 31 may allow a single-stage detachment of the punch tip 10 from the actuation device. If the slide of the actuation device is brought into a detachment position relative to its base body, the at least partial and substantial alignment of the contours of the attachment portions 21, 31 allows the punch tip 10 to be extracted laterally (i.e. upward in FIG. 1) from the base body and slide, without the slide blocking the first attachment portion 21.

For this, the second tip part 30 is suitably formed, in particular longitudinally displaceable relative to the first tip part 20, such that by bringing the slide into the detachment position, the second tip part 30 connected to the slide can also be brought into a detachment position relative to the first tip part 20, in which the contours of the attachment portions 21, 31 align with one another.

It is evident from a combined view of FIGS. 4 and 5 that the punch tip 10 may optionally have a centering device 11. Preferably, the centering device 11 is formed by the first step 28 and the second step (not shown in FIG. 5). To this end, the first step 28 may for example be formed as a disk, or at least partially have another convex form. The correspondingly formed second step can accordingly be oriented by pushing onto the first step 28, namely in that the proximal end of the second attachment portion 31 may, where applicable, be laterally deflected when stopping on the first step 28, because of the curvature of the latter. In other words, by creating a form fit between the first step 28 and the second step, in particular in the detachment position, the position of the proximal end of the second attachment portion 31 can be precisely defined. An alignment of the contours of the first and second attachment portions 21, 31 can thus be ensured.

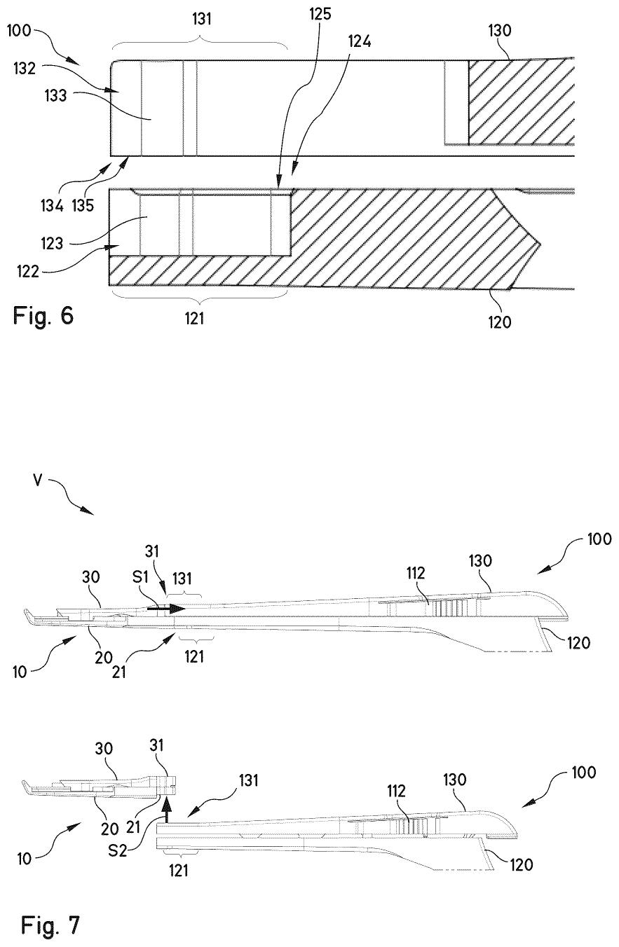

FIG. 6 shows an example of a distal end of an actuation device 100 in a side view. The actuation device 100 has a base body 120 and a slide 130 which is longitudinally displaceably attached to the base body 120. The slide 130 is here in the detachment position already shown in FIG. 2, in which it is raised from the base body 120 running parallel thereto. Thus FIG. 6 shows a base body top side 124 with a base body sliding face 125 of the base body 120, which faces a slide underside 134 of the slide 130 with a slide sliding face 135. The slide sliding face 135 is suitably configured to slide along the base body sliding face 125 on actuation of the actuation device 100.

The base body 120 has a base body attachment portion 121 for attachment of a first tip part of a medical punch tip (see FIGS. 3, 4 and 5). The base body attachment portion 121 may in particular be configured to receive a first attachment portion of the first tip part. For this, it is suitable for the base body attachment portion 121 to be configured, in particular formed, correspondingly to the first attachment portion. In particular, the base body attachment portion 121 may have a base body recess 122, into which a first protrusion of the first tip part (see FIGS. 4 and 5) can be inserted with form fit, i.e. which is formed correspondingly to the first protrusion.

The slide 130 has a slide attachment portion 131 for attachment of a second tip part of the punch tip (see FIGS. 3, 4 and 5). The slide attachment portion 131 may in particular be configured to receive a second attachment portion of the second tip part. For this, it is suitable for the slide attachment portion 131 to be configured, in particular formed, correspondingly to the second attachment portion. In particular, the slide attachment portion 131 may have a slide recess 132, into which a second protrusion of the second tip part (see FIGS. 4 and 5) can be inserted with form fit, i.e. which is formed correspondingly to the second protrusion.

For example, both the base body attachment portion 121 and also the slide attachment portion 131 may have an indentation 123, 133. These indentations 123, 133 may be configured to receive corresponding bulges of the first and second attachment portions (see FIG. 5).

As FIG. 6 clearly shows, the base body attachment portion 121 and the slide attachment portion 131 have at least partially substantially the same contour, in particular the same inner contour. For example, the base body sliding face 125 and the slide sliding face 135 may have at least partially the same contour.

In particular, it is suitable that—as shown in FIG. 6—the contours of the base body attachment portion 121 and slide attachment portion 131 at least partially substantially align with one another when the slide 130 is in the detachment position. In particular, the base body sliding face 125 and the slide sliding face 135 may at least partially align with one another. Thus a first attachment portion formed correspondingly to the base body attachment portion 121 can easily be conducted out of the base body attachment portion 121 through the slide attachment portion 131.

If the slide 130 is longitudinally displaced relative to the base body 120 out of the attachment position, the slide 130 can secure the first attachment portion received by the base body attachment portion 121. In other words, in such an operating position, the slide 130 may prevent the first attachment portion from being (undesirably) extracted from the base body attachment portion 121 through the slide attachment portion 131. This may be achieved for example if a part of the slide 130 at least partially overlaps the indentation 123 in the base body attachment portion 121.

FIG. 7 shows an example of a method V for detaching a medical punch tip 10 from an actuation device 100.

In a method step S1, a second tip part 30 of the punch tip 10, which is longitudinally displaceably mounted on a first tip part 20 of the punch tip 10 and is toollessly inseparably connected thereto, can be brought into a detachment position by longitudinal displacement relative to the first tip part 20 of the punch tip 10. Here, the second tip part 30 may in particular be longitudinally displaced such that a contour of a second attachment portion 31 of the second tip part 30, via which the second tip part 30 is attached to a slide 130 of the actuation device 100, aligns with a contour of a first attachment portion 21 of the first tip part 20, via which the first tip part 20 is attached to a base body 120 of the actuation device 100. For example, a protrusion of the second attachment portion 31 may be oriented in alignment with a protrusion of the first attachment portion 21.

The detachment position of the second tip part 30 is here preferably reached by longitudinal displacement of the slide 130 relative to the base body 120. The slide 130 may here be raised parallel to the base body 120.

In a further method step S2, then the punch tip 10 is laterally withdrawn from the actuation device 100. This is possible in particular since the first and second tip parts 20, 30 are toollessly inseparably connected together. Because the attachment portions 21, 31 align with one another, the first attachment portion 21 can be extracted from the actuation device 100 out of a base body attachment portion 121, in particular a base body recess, through a slide attachment portion 131, in particular a slide recess.

Accordingly, it is possible, for example by simple actuation of an actuation element 112 of the actuation device 100, to create a detachment position of the tip parts 20, 30 in which the punch tip 10 can be completely detached from the actuation device 100 with one manual action. This may mean a significant increase in user-friendliness, in particular acceleration of the tip change.

### LIST OF REFERENCE DESIGNATIONS

1 Surgical instrument
10 Punch tip
11 Centering device
20 First tip part
21 First attachment portion
22 First protrusion
23 Proximal end of first tip part
24 Top side
25 Top side face
25a Surface normal of top side face
26 Side wall
27 First bulge
28 First step
30 Second tip part
31 Second attachment portion
32 Second protrusion
33 Proximal end of second tip part
34 Underside
35 Underside face
35a Surface normal of underside face
36 Side wall
37 Second bulge
38 Second step
100 Actuation device
110 Actuation mechanism
111 Actuation lever
112 Actuation element
120 Base body
121 Base body attachment portion
122 Base body recess
123 Indentation
124 Base body top side
125 Base body sliding face
130 Slide
131 Slide attachment portion
132 Slide recess
133 Indentation
134 Slide underside
135 Slide sliding face
140 Handle
150 Shank
V Method
S1-S2 Method steps

1. A medical punch tip (10) for an actuation device (100), comprising:
a first tip part (20); and
a second tip part (30) mounted longitudinally displaceably on the first tip part (20),
wherein the second tip part (30) is toollessly inseparably connected to the first tip part (20).

2. The punch tip (10) as claimed in claim 1, wherein the first tip part (20) has a first attachment portion (21) for attachment of the first tip part (20) to a base body (120) of an actuation device (100), and the second tip part (30) has a second attachment portion (31) for attachment of the second tip part (30) to a slide (130) of the actuation device (100), wherein the first attachment portion (21) and the second attachment portion (31) have at least partially substantially the same contour.

3. The punch tip (10) as claimed in claim 2, wherein for toolless detachment of the punch tip (10) from the actuation device (100), the second tip part (30) can be brought into a detachment position by longitudinal displacement relative to the first tip part (20), in which position the contour of the second attachment portion (31) at least partially substantially aligns with the contour of the first attachment portion (21).

4. The punch tip (10) as claimed in claim 1, wherein:
the first tip part (20) has a top side (24) which faces the second tip part (30) and has a flat first sliding face,
the second tip part (30) has an underside (34) which faces the first tip part (20) and has a flat second sliding face, via which the second tip part (30) slides along the first tip part (20) during a longitudinal displacement, and
the first sliding face and the second sliding face are at least partially similarly formed.

5. The punch tip (10) as claimed in claim 1, wherein:
the first tip part (20) has a first protrusion (22) which, running longitudinally, adjoins a proximal end (23) of the first tip part (20) and has a top side (24) with a top side face (25),
the second tip part (30) has a second protrusion (32) which, running longitudinally, adjoins a proximal end (33) of the second tip part (30) and has an underside (34) with an underside face (35) which is oriented substantially parallel to the top side face (25), and
the first and second protrusions (22, 32) have respective side walls (26, 36) which run substantially perpendicularly to the top side face (25) and underside face (35) and are formed similarly uneven.

6. The punch tip (10) as claimed in claim 1, wherein:
the first tip part (20) has a first attachment portion (21) for attachment of the first tip part (20) to a base body (120) of the actuation device (100), and in the first attachment portion (21) has a top side (24) with a top side face (25) and a first bulge (27), which runs transversely to a surface normal (25a) of the top side surface (25), and
the second tip part (30) has a second attachment portion (31) for attachment of the second tip part (30) to a slide (130) of the actuation device (100), and in the second attachment portion (31) has an underside (34) with an underside face (35) facing the top side face (25) and a second bulge (37), which runs transversely to a surface normal (35a) of the underside surface (35) and is formed similarly to the first bulge (27).

7. The punch tip (10) as claimed in claim 6, wherein the first tip part (20) and the second tip part (30) each have a convex curvature in a region of the respective first or second bulge (27, 37).

8. The punch tip (10) as claimed in claim 1, wherein the first tip part (20) has a first attachment portion (21) for attachment of the first tip part (20) to a base body (120) of the actuation device (100), and in the first attachment portion (21) has a first step (28) which serves as a stop for the second tip part (30) on a longitudinal displacement.

9. The punch tip (10) as claimed in claim 8, wherein the second tip part (30) has a second attachment portion (31) for attachment of the second tip part (30) to a slide (130) of the actuation device (100), and in the second attachment portion (31) has a second step (38) formed correspondingly to the first step (28), such that a proximal end of the first attachment portion (21) substantially aligns with a proximal end of the second attachment portion (31) when the second tip part (30) stops on the first step (28).

10. The punch tip (10) as claimed in claim 1, wherein a centering device (11) is configured to orient the second tip part (30) relative to the first tip part (20) when, for detachment from an actuation device (100), the second tip part (30) is brought into a detachment position by longitudinal displacement relative to the first tip part (20).

11. The punch tip (10) as claimed in claim 1,
wherein the first tip part (20) has a first attachment portion (21) for attachment of the first tip part (20) to a base body (120) of the actuation device (100), and the second tip part (30) has a second attachment portion (31) for attachment of the second tip part (30) to a slide (130) of the actuation device (100), wherein the second attachment portion (31) protrudes laterally on both sides over the first attachment portion (21) transversely to a longitudinal extent of the punch tip (10).

12. A surgical instrument (1) having a medical punch tip (10) as claimed in claim 1 and an actuation device (100) for actuation of the punch tip (10).

13. A method (V) for detaching a medical punch tip (10) having a first tip part (20) and a second tip part (30), which is mounted longitudinally displaceably on the first tip part (20) and toollessly inseparably connected thereto, from an actuation device (100) having a base body (120) on which the first tip part (20) is attached, and a slide (130) which is longitudinally displaceably attached on the base body (120) and on which the second tip part (30) is attached, the method comprising:
(S1) bringing the second tip part (30) into a detachment position relative to the first tip part (20) by longitudinal displacement of the slide (130) relative to the base body (120); and
(S2) subsequently laterally withdrawing the punch tip (10) from the actuation device (100).
